(19) United States
(12) Patent Application Publication
(10) Pub. No.: US 2023/0420070 A1
(43) Pub. Date: Dec. 28, 2023

(54) Protein Structure Prediction

(71) Applicant: Microsoft Technology Licensing, LLC, Redmond, WA (US)

(72) Inventors: Tong Wang, Redmond, WA (US); Bin Shao, Beijing (CN); Tie-Yan Liu, Beijing (CN)

(21) Appl. No.: 18/038,333

(22) PCT Filed: Dec. 8, 2021
(86) PCT No.: PCT/US2021/062293

§ 371 Date: May 23, 2023

(30) Foreign Application Priority Data
Dec. 31, 2020 (CN) 202011631945.5

(51) Int. Cl. G16B 15/20; G16B 35/20

(52) U.S. Cl. CPC G16B 15/20; G16B 35/20

(57) ABSTRACT

According to implementations of the present disclosure, a solution is proposed for protein structure prediction. In this solution, from a fragment library for a target protein, a plurality of fragments is determined for each of a plurality of residue positions of the target protein. Each fragment comprises a plurality of amino acid residues. Then, a feature representation of structures of the plurality of fragments is generated for the each residue position. Next, a prediction of at least one of a structure and a structural property of the target protein is determined based on the respective feature representations generated for the plurality of residue positions. In this way, the solution can leverage structural information of fragment libraries to complement and complete information used in protein structure prediction, and the accuracy of protein structure prediction is thus improved.

### BACKGROUND

Proteins are biomolecules or macromolecules composed of long chains of amino acid residues. Proteins perform many significant life activities in organisms, and functions of proteins are mainly determined by their three-dimensional (3D) structures. Knowing the structures of proteins is very important to the fields of medicine and biotechnology. For example, if a certain protein plays a key role in a disease, drug molecules can be designed based on the structure of the protein to treat the disease. However, it is quite time-consuming to determine the structures of proteins through experiments, and there are only a small number of proteins whose structures are determined through experiments. Therefore, protein structure prediction at a low cost and with a high yield has become an important means for protein structure research.

### SUMMARY

According to implementations of the present disclosure, there is provided a solution for protein structure prediction. In this solution, a plurality of fragments is determined from a fragment library of a target protein for each of a plurality of residue positions for the target protein. Each fragment comprises a plurality of amino acid residues. Then, a feature representation of structures of the plurality of fragments is generated for the each residue position. Next, a prediction of at least one of a structure and a structural property of the target protein is determined based on the respective feature representations generated for the plurality of residue positions. In some implementations, the structure of the target protein may be predicted. In such implementations, structural information from fragment libraries can facilitate the search for a more realistic protein structure. In some implementations, a structural property of the target protein may be predicted. In such implementations, structural information from fragment libraries can improve the accuracy of predicting protein structural properties. In this way, the solution can leverage structural information of fragment libraries to complement and complete information used in protein structure prediction, and the accuracy of protein structure prediction is thus improved.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1 illustrates a block diagram of a computing device which can implement a plurality of implementations of the present disclosure;

FIG. 2 illustrates a schematic diagram of structural properties of a protein;

FIG. 3 illustrates a schematic diagram of a process of predicting a structure of a protein by using structural information of a fragment library according to some implementations of the present disclosure;

FIG. 4 illustrates a schematic diagram of a process of predicting structural properties of a protein by using structural information of a fragment library according to some implementations of the present disclosure;

FIG. 5 illustrates a schematic diagram of a process of encoding structural information of a fragment library by using a feature encoder according to some implementations of the present disclosure;

FIG. 6 illustrates a schematic diagram of a process of predicting structural properties of a protein by using a property predictor according to some implementations of the present disclosure; and

FIG. 7 illustrates a flowchart of a method for protein structure prediction according to implementations of the present disclosure.

Throughout the drawings, the same or similar reference signs refer to the same or similar elements.

### DETAILED DESCRIPTION

The present disclosure will now be discussed with reference to several example implementations. It is to be understood these implementations are discussed only for the purpose of enabling persons skilled in the art to better understand and thus implement the present disclosure, rather than suggesting any limitations on the scope of the subject matter.

As used herein, the term “includes” and its variants are to be read as open terms that mean “includes, but is not limited to.” The term “based on” is to be read as “based at least in part on.” The term “one implementation” and “an implementation” are to be read as “at least one implementation.” The term “another implementation” is to be read as “at least one other implementation.” The terms “first,” “second,” and the like may refer to different or same objects. Other definitions, explicit and implicit, may be included below.

As used herein, the term “neural network” can handle inputs and provide corresponding outputs and it generally includes an input layer, an output layer and one or more hidden layers between the input and output layers. The neural network used in the deep learning applications generally includes a plurality of hidden layers to extend the depth of the network. Individual layers of the neural network model are connected in sequence, such that an output of a preceding layer is provided as an input for a following layer, where the input layer receives the input of the neural network while the output of the output layer acts as the final output of the neural network. Each layer of the neural network includes one or more nodes (also referred to as processing nodes or neurons), each of which processes the input from the preceding layer. CNN is a type of neural network, including one or more convolutional layers for performing convolution operations on their respective inputs. CNN may be used in various scenarios and especially is suitable to process image or video data. In the text, the terms “neural network,” “network” and “neural network model” may be used interchangeably.

The structure of a protein is usually divided into a plurality of levels, including a primary structure, a secondary structure, a tertiary structure and so on. The primary structure refers to the arrangement order of amino acid residues, i.e., an amino acid sequence. The secondary structure refers to a specific conformation formed by main chain atoms along a certain axis, and includes α-helix, β-fold and random coil. The tertiary structure refers to a three-dimensional spatial structure formed through further coiling and folding of the protein on the basis of the secondary structure. A protein fragment (also referred to as a “fragment” for short) comprises a segment of continuous amino acid residues arranged in a three-dimensional spatial structure.

As mentioned above, the structure of a protein mainly affects its functionality, and protein structure prediction has become an important means for studying protein structure. Fragment assembly is an approach for protein structure prediction, and the quality of fragment libraries is a critical factor affecting the accuracy of fragment assembly. A fragment library is built based on fragments of a protein with a known structure (e.g., native fragments, near-native fragments). For a target protein of which the structure is to be predicted, different fragment library building algorithms may pick up as many native or near-native fragments as possible for each residue position (also referred to as “position) of the target protein.

The fragment library contains rich structural information, including but not limited to, secondary structures, torsion angles, distances and orientations between atoms. Although the fragment library is used in fragment assembly, the structural information contained in the fragment library has not yet been analyzed and leveraged. In addition, the structure prediction in fragment assembly is a Monte Carlo simulation process, which is very time-consuming.

Using gradient descent to fold a protein structure is another approach for protein structure prediction. In this approach, the protein structure is folded by optimizing potentials derived from predicted structural properties. The predicted structural properties may mainly comprise, for example, torsion angles and distances between C atoms and N atoms on the main chain. Given that the potentials are mainly derived from the predicted structural properties, the accuracy of the predicted structural properties, to a large extent, determines the quality of final predicted structures.

Currently, features widely used for protein structure prediction are those derived from protein amino acid sequences. That is, this approach only utilizes information of amino acid sequences but fails to exploit structural information contained in the fragment library.

In view of the above, according to implementations of the present disclosure, a solution for protein structure prediction is provided so as to solve the above problems and one or more of other potential problems. In the solution, a plurality of fragments is determined, from a fragment library for a target protein, for each of a plurality of residue positions of the target protein. Each fragment comprises a plurality of amino acid residues. Then, a feature representation of the structures of the plurality of fragments is generated for each residue position. Next, at least one of the structure and a structural property of the target protein is determined based on respective feature representations generated for the plurality of residue positions. In this way, the solution can leverage structural information of fragment libraries to complement and complete information used in protein structure prediction, and the accuracy of protein structure prediction is thus improved.

Various example implementations of the solution are described in detail below in conjunction with the drawings.

### Example Environment

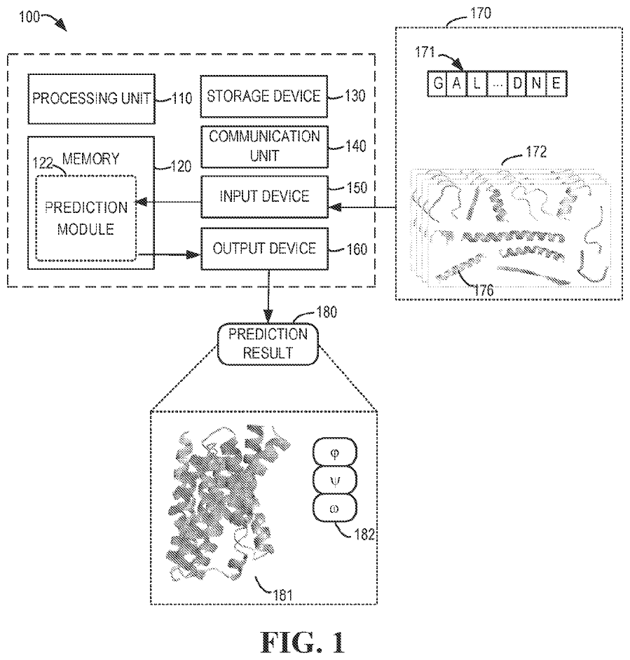

FIG. 1 illustrates a block diagram of a computing device 100 that can implement a plurality of implementations of the present disclosure. It should be understood that the computing device 100 shown in FIG. 1 is only exemplary and should not constitute any limitation on the functions and scopes of the implementations described by the present disclosure. As shown in FIG. 1, the computing device 100 includes a computing device 100 in the form of a general purpose computing device. Components of the computing device 100 may include, but is not limited to, one or more processors or processing units 110, a memory 120, a storage device 130, one or more communication units 140, one or more input devices 150, and one or more output devices 160.

In some implementations, the computing device 100 may be implemented as various user terminals or service terminals with computing capability. The service terminals may be servers, large-scale computing devices, and the like provided by a variety of service providers. The user terminal, for example, is a mobile terminal, a fixed terminal or a portable terminal of any type, including a mobile phone, a site, a unit, a device, a multimedia computer, a multimedia tablet, Internet nodes, a communicator, a desktop computer, a laptop computer, a notebook computer, a netbook computer, a tablet computer, a Personal Communication System (PCS) device, a personal navigation device, a Personal Digital Assistant (PDA), an audio/video player, a digital camera/video, a positioning device, a television receiver, a radio broadcast receiver, an electronic book device, a gaming device or any other combination thereof, including accessories and peripherals of these devices or any other combination thereof. It may also be predicted that the computing device 100 can support any type of user-specific interface (such as a “wearable” circuit, and the like).

The processing unit 110 may be a physical or virtual processor and may execute various processing based on the programs stored in the memory 120. In a multi-processor system, a plurality of processing units executes computer-executable instructions in parallel to enhance parallel processing capability of the computing device 100. The processing unit 110 can also be known as a central processing unit (CPU), microprocessor, controller and microcontroller.

The computing device 100 usually includes a plurality of computer storage mediums. Such mediums may be any attainable medium accessible by the computing device 100, including but not limited to, a volatile and non-volatile medium, a removable and non-removable medium. The memory 120 may be a volatile memory (e.g., a register, a cache, a Random Access Memory (RAM)), a non-volatile memory (such as, a Read-Only Memory (ROM), an Electrically Erasable Programmable Read-Only Memory (EEPROM), flash), or any combination thereof. The memory 120 may include sample processing modules 122, which are configured to perform various functions described herein. The sample processing module 122 may be accessed and operated by the processing unit 110 to realize corresponding functions.

The storage device 130 may be a removable or non-removable medium, and may include a machine-readable medium (e.g., a memory, a flash drive, a magnetic disk) or any other medium, which may be used for storing information and/or data and be accessed within the computing device 100. The computing device 100 may further include additional removable/non-removable, volatile/non-volatile storage mediums. Although not shown in FIG. 1, there may be provided a disk drive for reading from or writing into a removable and non-volatile disk and an optical disc drive for reading from or writing into a removable and non-volatile optical disc. In such cases, each drive may be connected to a bus (not shown) via one or more data medium interfaces.

The communication unit 140 implements communication with another computing device via a communication medium. Additionally, functions of components of the computing device 100 may be implemented by a single computing cluster or a plurality of computing machines, and these computing machines may communicate through communication connections. Therefore, the computing device 100 may operate in a networked environment using a logic connection to one or more other servers, a Personal Computer (PC) or a further general network node.

The input device 150 may be one or more various input devices, such as a mouse, a keyboard, a trackball, a voice-input device, and the like. The output device 160 may be one or more output devices, e.g., a display, a loudspeaker, a printer, and so on. The computing device 100 may also communicate through the communication unit 140 with one or more external devices (not shown) as required, where the external device, e.g., a storage device, a display device, and so on, communicates with one or more devices that enable users to interact with the computing device 100, or with any device (such as a network card, a modem, and the like) that enable the computing device 100 to communicate with one or more other computing devices. Such communication may be executed via an Input/Output (I/O) interface (not shown).

In some implementations, apart from being integrated on an individual device, some or all of the respective components of the computing device 100 may also be set in the form of a cloud computing architecture. In the cloud computing architecture, these components may be remotely arranged and may cooperate to implement the functions described by the present disclosure. In some implementations, the cloud computing provides computation, software, data access and storage services without informing a terminal user of physical locations or configurations of systems or hardware providing such services. In various implementations, the cloud computing provides services via a Wide Area Network (such as Internet) using a suitable protocol. For example, the cloud computing provider provides, via the Wide Area Network, the applications, which can be accessed through a web browser or any other computing component. Software or components of the cloud computing architecture and corresponding data may be stored on a server at a remote location. The computing resources in the cloud computing environment may be consolidated at a remote datacenter or dispersed. The cloud computing infrastructure may provide, via a shared datacenter, the services even though they are shown as a single access point for the user. Therefore, components and functions described herein can be provided using the cloud computing architecture from a service provider at a remote location. Alternatively, components and functions may also be provided from a conventional server, or they may be mounted on a client device directly or in other ways.

The computing device 100 may be used for implementing protein structure prediction in various implementations of the present disclosure. As shown in FIG. 1, the computing device 100 may receive through the input device 150 input information 170 related to a target protein of which the structure is to be predicted. The input information 170 may comprise an amino acid sequence 171 of the target protein, which indicates types and arrangement order of amino acids forming the target protein. The input information 170 may further comprise a fragment library 172 for the target protein. The fragment library 172 may assign a plurality of fragments with known structures to each residue position of the target protein, such as a fragment 176. As used herein, a residue position (also referred to as “position” for short) of the target protein corresponds to an amino acid residue in the target protein. A fragment assigned by the fragment library to the residue position is also referred to as “a template fragment”. Such a template fragment is usually composed of a plurality of amino acid residues (e.g., 7 to 15 amino acid residues), thereby containing structural information of these amino acid residues.

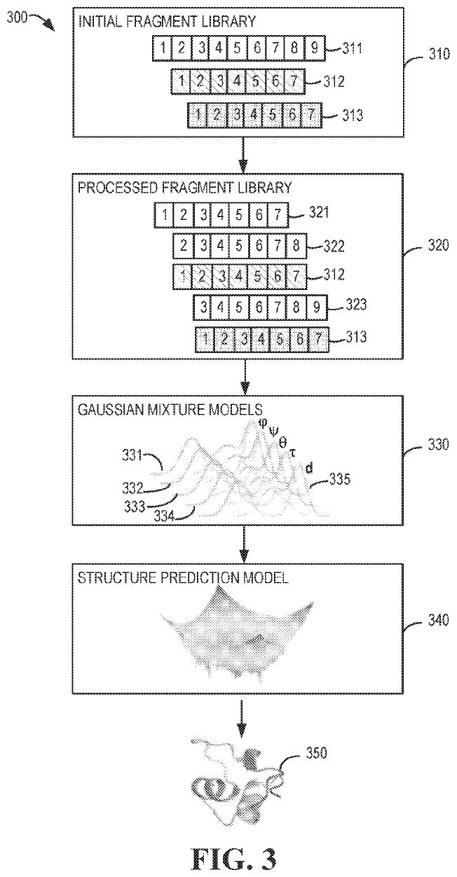

These fragments assigned by the fragment library 172 are selected from a large number of fragments obtained by cutting proteins with known structures using a fragment library building algorithm. The fragment library 172 may be built for the target protein based on any appropriate fragment library algorithm. Appropriate fragment library building algorithms may comprise, but are not limited to, NNMake, LRFragLib, Flib-Coevo, and DeepFragLib, etc. In some implementations, the fragment library 172 may be an initial fragment library (such as a fragment library 310 shown in FIG. 3) built for the target protein using a fragment library building algorithm. In some implementations, the fragment library 172 may be a processed fragment library (such as a processed fragment library 320 shown in FIG. 3) derived from processing an initial fragment library.

In some implementations, different fragment library building algorithms may be evaluated using reference proteins with known structures. Then, an algorithm for constructing the fragment library 172 may be selected from the different fragment library building algorithms based on the evaluation, as will be detailed below.

The computing device 100 (e.g., a prediction module 122) may extract structural information of the fragment library 172, e.g., one or more structural properties of an assigned fragment. The computing device 100 further may provide a prediction result 180 related to the structure of the target protein based on the extracted structural information. In some implementations, the prediction result 180 may include a prediction 181 of the structure of the target protein, e.g., including a spatial coordinate representation of main atoms of the target protein. Alternatively, or in addition, in some implementations, the prediction result 180 may include a prediction 182 of structural properties of the target protein, e.g., a prediction of torsion angles φ, ψ and ω.

Although in the example shown in FIG. 1, the computing device 100 receives the input information 170 from the input device 150 and provides the prediction result 180 via the output device 160, this is merely illustrative without any limitation to the scope of the present disclosure. The computing device 100 may further receive the input information 170 from other devices (not shown) via the communication unit 140 and/or provide the prediction result 180 externally via the communication unit 140. In addition, in some implementations, the computing device 100 may construct the fragment library 172 for the target protein by using a fragment library building algorithm, instead of obtaining a built fragment library.

### Structural Properties of Proteins and Fragments

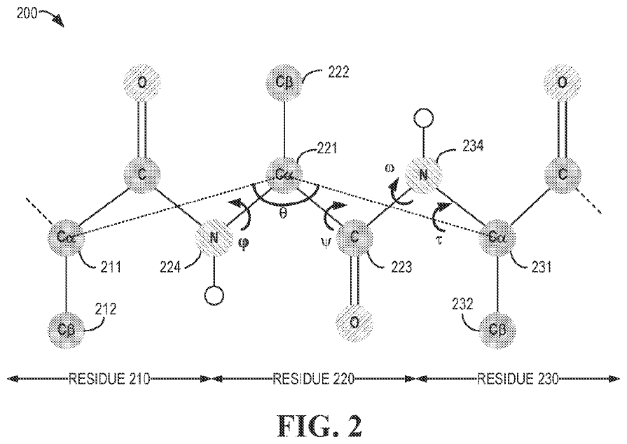

As mentioned above, the implementations of the present disclosure extract structural information, e.g., various structural properties of fragments, from the fragment library 172. In addition, in some implementations of the present disclosure, a structural property of the target protein may be predicted. To better understand the implementations of the present disclosure, reference is made to FIG. 2 to describe structural properties of proteins. A fragment 200 shown in FIG. 2 comprises residues 210, 220 and 230. Each residue comprises N atoms, Cα atoms and C atoms on the main chain, as well as Cβ atoms and O atoms on side chains.

Structural properties of a protein may comprise inter-residue distances between a plurality of resides. Inter-residue distances may comprise distances between the same type of atoms in two resides, such as a Cα-Cα distance and a Cβ-Cβ distance. The Cα-Cα distance refers to a distance between pairwise Cα-Cα atoms (also referred to as an inter-residue Cα distance). The Cα-Cα distance may comprise a distance between a pair of neighboring Cα atoms or a distance between a pair of any non-neighboring Cα atoms, such as a distance between any two of Cα atoms 211, 221 and 231 in FIG. 2. The Cβ-Cβ distance refers to a distance between pairwise Cβ-Cβ atoms (also referred to as ab inter-residue Cβ distance). The Cβ-Cβ distance may comprise a distance between a pair of neighboring Cβ atoms or a distance between a pair of any non-neighboring Cβ atoms, such as a distance between any two of Cβ atoms 212, 222 and 232 in FIG. 2.

The structural properties of the protein may further comprise inter-residue orientations between a plurality of resides. Inter-residue orientations may comprise an angle between a plurality of atoms in two resides, such as torsion angles φ and ω, backbone angles θ and τ, etc. The torsion angle φ refers to a dihedral angle for an N-Cα chemical bond. The torsion angle ω refers to a dihedral angle for a chemical bond C—N. For examples, with respect to the residues 220 and 210, the torsion angle φ is a dihedral angle for a chemical bond between the N atom 224 and the Cα atom 221. With respect to the residues 220 and 230, the torsion angle in is a dihedral angle for a chemical bond between the C atom 223 and the N atom 234. The backbone angle θ refers to a dihedral angle for a Cα-Cα-Cα chemical bond of neighboring residues. The backbone angle τ refers to a dihedral angle for a Cα-Cα chemical bond of neighboring residues. For example, with respect to the residue 220, the backbone angle θ is the angle, at the Cα atom 221, of the triangle formed by its Cα atom 221 and the Cα atoms 211 and 231 in the neighboring residues 210 and 230, and the backbone angle τ is a dihedral angle of the line between the Cα atom 221 and the Cα atoms 231 (or 211).

Structural properties of the protein may further comprise other orientations between atoms of the protein. For example, structural properties may further comprise a torsion angle ψ within residues as shown in FIG. 2. The torsion angle ψ refers to a dihedral angle for a Cα-C chemical bond within a residue. For example, with respect to the residue 220, the torsion angle ψ is a dihedral angle for a chemical bond between the Cα atom 221 and the C atom 223. In addition, structural properties of the protein may further comprise bond lengths and bond angles between continuous atoms on the main chain. Bond lengths may comprise a bond length between N—Cα atoms within a residue, a bond length between Cα-C atoms within a residue, a bond length between C—N atoms within a residue, etc. Bond angles may comprise bond angles between N—Cα-C atoms within a residue, between Cα-C—N atoms of residues, and between C—N—Cα atoms of residues, etc. Among structural properties described above, the torsion angles φ, ψ and ω represent angles between different types of atoms, and the backbone angles θ and τ represent angles between the same type of atoms.

The structural properties as described above are defined at the level of the amino acid residues. As mentioned above, the fragment comprises a segment of continuous amino acid residues arranged in a three-dimensional structure. Therefore, it is to be understood that a fragment can also have the structural properties as described above, such as the Cα-Cα distance, the Cβ-Cβ distance, the torsion angle φ, ψ, ω, the backbone angles θ, τ, etc.

Besides those structural properties as described above, the structural properties of the fragment may further comprise a secondary structure. The secondary structure of a fragment may be divided into four classes: mainly helix (termed as H), mainly fold (termed as E), mainly coil (termed as C) and others (termed as O). A fragment is defined as H or E or C if more than half the residues of the fragment have the corresponding secondary structures. Otherwise, the secondary structure of the fragment is defined as O.

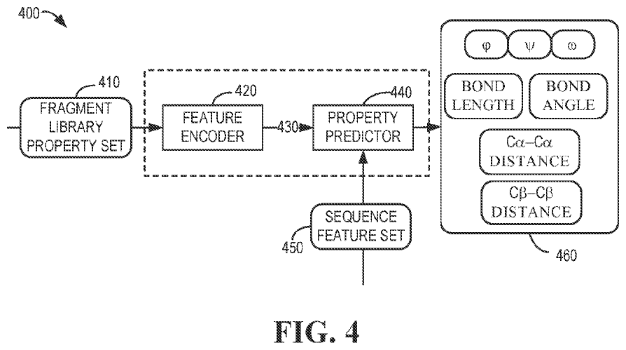

In some implementations, the computing device 100 may extract one or more of the above structural properties from the fragments assigned from the fragment library 172 to predict the structure of the target protein, as to be described with reference to FIG. 3. In some implementations, the computing device may predict one or more of the structural properties of the target protein by using the structural properties extracted from the fragment library 172, as to be described with reference to FIGS. 4 to 6.

### Evaluation of Fragment Library

Fragment libraries built by different fragment library building algorithms (also abbreviated as “algorithms” herein) might have different performance. In some implementations, performance of fragment libraries built by different algorithms may be evaluated using evaluation metrics. Specifically, different algorithms may be used to construct a plurality of reference fragment libraries for a reference protein, of which the structure is known. Then, for each reference fragment library, property values (also referred to as “reference property values”) of a structural property of a plurality of reference fragments assigned by each reference fragment library to a reference residue position of the reference protein may be determined, and property values (also referred to as “reference property values”) of the structural property at the reference residue position of the reference protein may be determined. A difference between the reference property values and a true property value of the same structural property may be used as an evaluation metric. Evaluation metrics for evaluating fragment libraries built by different algorithms usually comprise precision and coverage. Precision is the proportion of good fragments in the whole fragment library and coverage is the proportion of positions which are spanned by at least one good fragment, where a good fragment is a fragment whose root-mean-square error (RMSD) with respect to the true fragment at a position is lower than a given RMSD. Therefore, a good fragment may be a fragment with a similarity exceeding a threshold similarity to a true fragment at the position.

As classical metrics, precision and coverage fail to reflect the accuracy of structural properties of fragments. To this end, in some implementations of the present disclosure, evaluation metrics related to structural properties may be used to make a comprehensive evaluation on the fragment libraries. Such structural properties may comprise, for example, the secondary structures, the torsion angles φ, ψ, ω, the backbone angles θ, τ, and the pairwise Cα-Cα and Cβ-Cβ distances, etc. In the implementations of the present disclosure, an evaluation metric may be defined as the accuracy or error of these structural properties at the fragment level.

In some implementations, the evaluation metrics may comprise the accuracy of the secondary structure at the fragment level. As described above, the secondary structure of a fragment may be divided into H, E, C and O. Therefore, the accuracy of the secondary structure at the fragment level may be expressed as:

$${I}_{SS} ( {f}_{i} ) = \{ \begin{matrix}1 , if SS ( {f}_{i} ) = SS ( {f}_{*} ) \\ 0 , otherwise\end{matrix} \qquad ( 1 )$$

$$ACCss ( FL ) = {E}_{pi} [ {E}_{fi} [ {I}_{SS} ( {f}_{i} ) ] ] \qquad ( 2 )$$

where FL denotes the fragment library, E denotes the mathematical expectation, pᵢ denotes all fragments at position i (i.e., all fragments assigned by the fragment library to position i), fᵢ denotes a fragment at position i, f, denotes the corresponding true fragment of the target protein and SS(f) denotes the secondary structure of fragment f Thus, the accuracy of the secondary structure of the whole fragment library ACC_{SS}(FL) is defined as the expectation of the accuracy of all positions, where the accuracy of each position is then defined as the expectation of the accuracy of all template fragments at the position.

Alternatively, or in addition, in some implementations, the evaluation metrics may comprise the error of structural properties at the fragment level, e.g., the error of angles φ, ψ, ω, θ and τ. The error of angles φ, ψ, ω, θ and τ may be expressed as:

$${err}_{ang} ( {f}_{i} , {f}_{*} ) = \frac{1}{N} {\sum }_{j = 1}^{N} min \{ \vert {ang}_{i}^{j} - {ang}_{*}^{j} \vert , 360 ° - \vert {ang}_{i}^{j} - {ang}_{*}^{j} \vert \} \qquad ( 3 )$$

$${ERR}_{ang} ( FL ) = {E}_{pi} [ {E}_{fi} [ {err}_{ang} ( {f}_{i} , {f}_{*} ) ] ] \qquad ( 4 )$$

where ang denotes any of φ, ψ, ω, θ and τ, |x| denotes the absolute value of x, angᵢ^{j} denotes the angle value of residue j of the fragment fᵢ, N denotes the number of residues of the fragment fᵢ, ang_{*}^{j} denotes the true angle value of the corresponding residue j in the reference protein and err_{ang}(fᵢ, f_{*}) denotes the mean absolute error (MAE) of the angle of the fragment fᵢ. Thus, the error of angles φ, ψ, ω, θ and τ may be defined as the expectation of angle errors of all positions, where the angle error of each position is then defined as the expectation of angle errors of all template fragments at the position.

Alternatively, or in addition, in some implementations, the evaluation metrics may comprise the error of inter-residue distances, e.g., the error of Cα-Cα distances and the error of Cβ-Cβ distances. The error of Cα-Cα distances and the error of Cβ-Cβ distances may be expressed as:

ERR_{dist}(FL)=Eₚᵢ[E_{fi}[err_{dist}(fᵢ,f_{*})]] (5)

where err_{dist}(fᵢ, f_{*}) denotes the MAE of the Cα-Cα distance or Cβ-Cβ distance within a fragment fᵢ as compared with the true Cα-Cα distance or Cβ-Cβ distance within a corresponding fragment f_{*} of the reference protein.

With reference to Equations (1) to (5), description has been presented above to the evaluation metrics related to structural properties at the fragment level, including the accuracy of the secondary structure, the errors of angles φ, ψ, ω, θ and τ, the error of Cα-Cα distances and the error of Cβ-Cβ distances.

In some implementations, one or more of these evaluation metrics may be used to evaluate fragment libraries built using different algorithms. Fragment libraries with a higher accuracy of the secondary structure and a smaller error of angles or distances may be considered to have better performance.

In some implementations, an algorithm may be selected based on the evaluation of fragment libraries built by different algorithms, so as to build the fragment library 172 for the target protein. For example, a plurality of reference fragment libraries may be built for the reference protein using different algorithms. Then, for each reference fragment library, reference property values, e.g., angᵢ^{j} in Equation (3), of a structural property of the plurality of reference fragment libraries assigned by each reference fragment library at a reference residue position of the reference protein may be determined. Since the reference protein has a known structure, the true property value, e.g., ang_{*}^{j} in Equation (3), of the structural property of the reference protein at the reference residue position may be determined. Next, a difference between the reference property values and the true property value may be determined, for example, the error may be calculated according to Equation (4). Finally, an algorithm may be selected based on the differences determined for the plurality of reference fragment libraries.

As an example, fragment libraries FA, FB and FC may be built for the reference protein according to algorithms A, B and C, respectively. Then, for each of the fragment libraries FA, FB and FC, the evaluation metrics defined by Equations (2), (4) and (5) may be calculated, respectively. If the performance of the fragment library FA is superior to the performance of the fragment libraries FB and FC in terms of evaluation metrics exceeding a threshold number (e.g., 3) among these evaluation metrics, the algorithm A may be selected to build the fragment library 172 for the target protein.

In such implementations, a comprehensive evaluation on structural information contained in fragment libraries may be made by using evaluation metrics at the fragment level, so that performance of different fragment library building algorithms may be evaluated. In this way, a fragment library building algorithm with better performance may be selected to build the fragment library for the target protein. This helps improve the accuracy of protein structure prediction or structural property prediction.

### Prediction of Protein Structure

In some implementations, the structure of the target protein may be predicted using structural information of the fragment library 172 for the target protein. For example, for each residue position, the prediction module 122 may determine a property value of a structural property of each fragment assigned to the residue position, such a structural property may be, for example, one or more of angles φ, ψ, ω, θ, τ, the Cα-Cα distance and the Cβ-Cβ distance. Then, the prediction module 122 may determine for each residue position of the target protein a feature representation of the considered structural property, e.g., a probability distribution. The prediction module 122 may predict the structure of the protein based on the feature representation of the structural property.

The example process of predicting protein structure will be described by taking angles φ, ψ, ω, θ, τ, the Cα-Cα distance and the Cβ-Cβ distance as examples of the structural property. However, it should be understood this is merely exemplary without any limitation the scope of the present disclosure, and the protein structure may be predicted based on other structural properties.

FIG. 3 shows a schematic diagram of a process 300 of predicting the structure of a protein by using structural information of a fragment library according to some implementations of the present disclosure. In the example of FIG. 3, the prediction module 122 may extract from a fragment library for the target protein a plurality of structural properties of each fragment, including angles φ, ψ, ω, θ, τ, the Cα-Cα distance and the Cβ-Cβ distance, etc.

An initial fragment library 310 built by a fragment library building algorithm may assign a plurality of initial fragments to each position, e.g., fragments 311, 312 and 313. As shown in FIG. 3, lengths of the initial fragments might vary. The “length of a fragment” as described herein refers to the number of amino acid residues included in the fragment. For example, the fragment 311 has 9 amino acid residues, and its length is 9; the fragment 312 has 7 amino acid residues, and its length is 7; and the fragment 313 has 7 amino acid residues, and its length is 7.

In some implementations, the prediction module 122 may obtain a processed fragment library 320 by processing the initial fragment library 310. In the processed fragment library 320, a plurality of fragments assigned to the same position may have the same length. The prediction module 122 may generate a fragment with a predetermined number of residues from an initial fragment in the initial fragment library 310. As an example, the prediction module 122 may perform a smoothing operation on a fragment whose length exceeds a threshold. The smoothing operation may cut the initial fragment into a series of fragments each including the predetermined number of residues by a sliding window. The smoothing operation may result in a situation where all fragments assigned to the same position have the same length. In the example of FIG. 3, the sliding window of the smoothing operation has a length of 7. Accordingly, the prediction module 122 may generate fragments 321, 322 and 323 with a length of 7 from the initial fragment 311 with a length of 9. It may be understood that the lengths of fragments in the processed fragment library 320 shown in FIG. 3 are merely exemplary without any limitation to the scope of the present disclosure. In the implementations of the present disclosure, fragments assigned to a residue position may be processed to have any appropriate length.

Then, the prediction module 122 may determine, for each residue position based on structures of a plurality of fragments assigned to the position, the probability distribution of the structural property at the residue position as the feature representation of the structural property. In the example of FIG. 3, the prediction module 122 may determine the probability distributions of angles φ, ψ, ω, θ, τ, the Cα-Cα distance dα and the Cβ-Cβ distance dβ for each residue position.

Description is presented to how to use Gaussian mixture models to delineate the probability distributions of the structural properties at each residue position. However, it should be understood that this is merely exemplary without any limitation to the scope of the present disclosure, and any appropriate models may be employed to delineate the probability distributions of the structural properties in the implementations of the present disclosure.

Some fragments of the plurality of fragments assigned by the fragment library 320 to the residue position i might be good fragments, while others might not be good fragments. As mentioned above, RMSD may be used to evaluate whether a fragment is a good one or not. Given that each fragment assigned by the fragment library 320 may have a predicted RMSD value, the predicted RMSD value may be regarded as a confidence score for the fragment. For example, the prediction module 122 may assign a weight w_{fi} to each fragment at the same residue position i according to the following equation:

$${w}_{fi} = \frac{{e}^{( 5. - {predRMSD}_{i} )} / T}{{\sum }_{{f}_{j}}^{F} {e}^{( 5. - {predRMSD}_{i} )} / T} \qquad ( 6 )$$

where F denotes all fragments at the same residue position i, fᵢ denotes a fragment in the set F of fragments, and predRMSDᵢ denotes the predicted RMSD value of fragment fᵢ and T is the temperature.

Equation (7) shows a probability density function of a Gaussian distribution:

$$p ( y ; μ ; σ ) = \frac{1}{\sqrt{2 π \vert σ \vert}} exp ( - \frac{{( y - μ )}^{2}}{2 {σ}^{2}} ) \qquad ( 7 )$$

where y is the property value of a structural property weighted by w_{fi} in the Equation (6), μ and σ² denote the mean and variance, respectively.

Then, the prediction module 122 may build weighted Gaussian mixture models (wGMMs) 330 of each structural property for each residue position. The weighted Gaussian mixture models 330 may have any appropriate number of components. Components refer to the number of Gaussian distributions in the weighted Gaussian mixture model. In the implementations of the present disclosure, weighted Gaussian mixture models built for different residue positions may have the same or a different number of components. In the example of FIG. 3, fragments assigned to each residue position have a length of 7, i.e., having 7 residues. Therefore, for each residue position, the prediction module 122 may build 7 wGMMs for each of angles φ, ψ, ω, θ and τ, and build 21 wGMMs for each of the Cα-Cα distance dα and the Cβ-Cβ distance dβ, thereby resulting in 70 wGMMs in total. In the example of FIG. 3, a Gaussian distribution 331 of angle φ, a Gaussian distribution 332 of angle ψ, a Gaussian distribution 333 of angle θ, a Gaussian distribution 334 of angle τ, and a Gaussian distribution 335 of distance d (any of the Cα-Cα distance dα and the Cβ-Cβ distance dβ).

In this way, the prediction module 122 may determine, for each residue position, the Gaussian distribution of a considered structural property at the residue position as a feature representation, which is also referred to as “a first feature representation” herein. Then, the prediction module 122 may generate a potential function corresponding to the structural property based on the Gaussian distribution at the plurality of residue positions of the target protein.

In some implementations, the Gaussian distribution may be converted to the potential function by using a negative log likelihood function. It may be understood that since the wGMM is specific to the target protein, the potential function derived from fragments as such is customized for the target protein. Equations (8) and (9) show examples of potential functions of structural properties:

$${L}_{φ} ( x ) = - {\sum }_{fi} log {\sum }_{m} {\sum }_{t = 1}^{K} {w}_{i , t , m}^{φ} p ( {φ}_{i} ; {μ}_{i , t}^{φ} ; {σ}_{i , t}^{φ} ) \qquad ( 8 )$$

$$( 9 )$$

$${L}_{φ} ( x ) = - {\sum }_{{f}_{i}} {\sum }_{{j}_{1} < {j}_{2}} log {\sum }_{n} {\sum }_{t = 1}^{K} {w}_{i , {j}_{1} , {j}_{2} , t , n}^{{C}_{β}} p ( {d}_{{j}_{1} , {j}_{2}}^{{C}_{β}} ; {μ}_{i , {j}_{1} , {j}_{2} t}^{{C}_{β}} ; {σ}_{i , {j}_{1} , {j}_{2} , t}^{{C}_{β}} )$$

where Equation (8) is the potential function corresponding to angle φ, Equation (9) is the potential function corresponding to the Cβ-Cβ distance, x denotes a predicted structure of the target protein, K is the number of components in wGMM, w, μ and σ are the fitted parameters of each component in wGMM, φᵢ is the angle φ at the i-th residue in the structure x, fᵢ denotes a fragment assigned for the i-th residue, m denotes the number (e.g., 7 as mentioned above) of wGMMs built for angle φ at the i-th residue, dⱼ_{₁}_{,j}_{₂}^{c}^{_{β}} is the distance between the atom Cβ of the j₁-th residue and the atom Cβ of the j₂-th residue in the fragment fᵢ, and n denotes the number (e.g., 21 as mentioned above) of wGMMs built for the Cβ-Cβ distance of the i-th residue. The potential functions corresponding to angles ψ, θ and τ may be defined in a way similar to Equation (8), and the potential function corresponding to the Cα-Cα distance may be defined in a way similar to Equation (9). In this way, where six structural properties are extracted from fragments, six potential functions may be defined in total, one for each structural property.

After determining potential functions corresponding to the plurality of structural properties respectively, the prediction module 122 may determine a target function for a structure prediction model 340 based on the determined potential functions. The structure prediction model 340 may be configured to predict the structure of a protein by minimizing the target function. For example, the structure prediction model 340 may be a gradient descent-based protein folding framework.

In the case where the considered structural properties comprise angles φ, ψ, θ and τ, the Cα-Cα distance da and the Cβ-Cβ distance dβ, a combined potential function may be expressed as:

L_{FL}(x)=w_{φ}L_{φ}(x)+w_{ψ}L_{ψ}(x)+w_{θ}L_{θ}(x)+w_{τ}L_{τ}(x)+w_{C}_{_{α}}L_{C}_{_{α}}(x)+w_{C}_{_{β}}L_{C}_{_{β}}(x) (10)

where L_{FL}(x) is defined as the weighted sum of the six potential functions, and L_{φ}(x) L_{ψ}(x), L_{θ}(x), L_{τ}(x), L_{Cα}(x) and L_{Cβ}(x) denote the potential function of angles φ, ψ, θ and τ, the Cα-Cα distance da and the Cβ-Cβ distance dβ respectively, w_{φ}, w_{ψ}, w_{θ}, w_{τ}, w_{Cα} and w_{Cβ} denote the weights for potential functions of angles φ, ψ, θ and τ, the Cα-Cα distance da and the Cβ-Cβ distance dβ respectively. Weights in Equation (10) may be regarded as hyper-parameters and may be tuned on a reference dataset (e.g., CASP12FM), which comprises information of reference proteins with known structures. For example, the weights in Equation (10) may be tuned on the reference dataset by maximizing the mean template modeling (TM) score of predicted structures.

The combined potential function shown in Equation (10) may be used as a portion of the target function. The target function may further comprise one or more geometric potential functions for constraining the geometric structure of the target protein, so that the predicted structure is a biophysically reasonable structure. As such, the prediction module 122 may determine the target function for the structure prediction model 340. Next, the prediction model 122 may generate a predicted structure 350 of the target protein by minimizing the target function according to the structure prediction model 340. For example, the prediction model 122 may calculate and minimize the target function in each step of the gradient descent process so as to update the structure of the target protein.

Description has been presented above to example implementations of predicting the structure of a protein by using structural information of the fragment library. In such implementations, probability distributions of structural properties are used to explicitly represent structural features of the fragments in the fragment library, and protein-specific potential functions are determined based on the probability distributions. Such potential functions derived from the fragment library may be subsequently used for the structure prediction model, e.g., the gradient descent-based protein folding model, so as to predict the structure of the protein. This method which uses potential functions derived from the fragment library outperforms methods which do not use potential functions derived from the fragment library in several aspects (for example, the mean TM score of decoys and the number of decoys with TM scores greater than 0.5). Therefore, structural information of the fragment library can facilitate the structure prediction model to seek a more realistic structure for the target protein.

### Prediction of Protein Structural Properties

In the implementations described above, explicit representations of structural information of the fragment library are used to predict the structure of a protein. Alternatively, or in addition, in some implementations, structural information of the fragment library 172 for the target protein may be utilized to predict structural properties of the target protein. For example, for each residue position, the prediction module 122 determines a plurality of structural properties (e.g., two or more of angles φ, ψ, ω, bond lengths, and bond angles) of each fragment of a plurality of fragments assigned to the residue position. Then, the prediction module 122 may encode a plurality of structural properties determined for the plurality of fragments according to a trained feature encoder, so as to determine feature representations of structures of the plurality of fragments. The prediction module 122 may predict a structural property of the target protein based on a feature representation (also referred to as “a second feature representation” herein) of an amino acid sequence and feature representations determined for each residue position.

FIG. 4 shows a schematic diagram of a process 400 of predicting structural properties of a protein by using structural information of a fragment library according to some implementations of the present disclosure. In the example of FIG. 4, a fragment library property set 410 is first extracted from the fragment library 172. Specifically, for each residue position of the target protein, the prediction module 122 may select a predetermined number F of fragments from fragments assigned by the fragment library 172 to the residue position, where F is a positive integer, e.g., 50. For example, the prediction module 122 may select F fragments with the lowest predicted RMSD values from the assigned fragments. The prediction module 122 may extract a plurality of structural properties of each of F fragments for each residue position, for example, the one-hot code of the residue secondary structure (such as “0001” denotes H, “0010” denotes E, “0100” denotes C, “1000” denotes the sine and cosine values of torsion angles φ, ψ, ω, bond lengths between C—N, N—Cα and Cα-C atoms of each residue, and bond angles of Cα-C—N, C—N—Cα and N—Cα-C of each residue, etc. If F fragments have different lengths, then all F fragments may be padded to have a length of a predetermined number R of residues, where R is a positive integer, e.g., As such, the prediction module 122 may determine the fragment library property set 410 from the fragment library 172 for the target protein. The fragment library property set 410 may be represented as an L×F×R×D tensor, where L denotes the length of the target protein, i.e., the number of amino acid residues, and D denotes the dimension of structural properties extracted from the fragments.

The fragment library property set 410 may be subsequently inputted to a trained feature encoder 420. The feature encoder 420 may generate a fragment library feature set 430 by encoding the fragment library property set 410. The fragment library feature set 430 may comprise encoded structural properties for each residue position. That is, for each residue position, the feature encoder 420 may obtain a structural feature at the residue position based on the structural properties of the plurality of fragments.

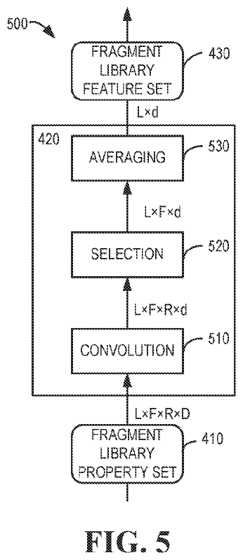

Reference is made to FIG. 5, which shows a schematic diagram of a process 500 of encoding structural information of a fragment library by using the feature encoder 420 according to some implementations of the present disclosure. As shown in FIG. 5, the feature encoder 420 has a hierarchical architecture which comprises three levels of encoding processes. First, in a convolution process 510, the fragment library property set 410 represented by an L×F×R×D tensor is convolved. As an example, each building block constituting the convolutional network may include two convolution layers for performing convolutional operations on the third dimension of the input L×F×R×D tensor (the dimension of fragment length). In addition, an exponential linear unit (ELU) activation layer may be adopted between the two convolutional layers. The two convolutional layers may have a convolutional kernel with any appropriate size and any appropriate number of filters. If d filters are used in the convolution process 510, the dimension of the tensor outputted by the convolution process 510 is L×F×R×d, as shown in FIG. 5. The convolution process 510 functions to learn interactions between neighboring residues within a fragment. To this end, a certain number (e.g., 8) of building blocks may be stacked with skip connections. The above described convolution process 510 is merely exemplary without any limitation to the scope of the present disclosure. In the implementations, the convolution process 510 may be implemented in any appropriate way.

After performing the convolution process 510, the plurality of structural properties is converted into implicit representations. Next, in a selection process 520, for each fragment at each residue position, the implicit representation of one residue of the fragment may be selected. For example, given that the index of the first residue of the fragment corresponds to the residue position of the target protein, the implicit representation of the first residue of each fragment may be selected. As such, the dimension of a feature map outputted by the selection process 520 is L×F×d, as shown in FIG. 5.

Finally, in an averaging process 530, an output tensor with L×d dimension may be obtained as the fragment library feature set 430, by averaging all the F fragments at the same residue position. A 1×d vector corresponding to each residue position in the fragment library feature set 430 may be regarded as the feature representation of the fragment determined for the residue position.

Reference is made back to FIG. 4. The fragment library feature set 430 with L×d dimension is inputted into a trained property predictor 440. The property predictor 440 also receives a sequence feature set 450 of an amino acid sequence of the target protein. The sequence feature set 450 may comprise at least one of: an elementary sequence of the target protein, the Position-Specific Scoring Matrix (PSSM) of homologous proteins and the pairwise statistics derived from direct coupling analysis (DCA). For example, the fragment library feature set 430 outputted by the feature encoder 420 as well as the one-hot codes of the elementary sequence of the target protein and PSSM may be transformed into a two-dimensional representation by tiling both horizontally and vertically, which is then concatenated with the pairwise statistics to form the total input of the property predictor 440. The property predictor 440 is trained to predict structural properties 460 of the target protein based on feature representations of the fragments in the fragment library and the feature representation of the amino acid sequence. The predicted structural properties 460 comprise for example torsion angles φ, ψ, ω, bond lengths between C—N, N—Cα and Cα-C atoms of each residue, and bond angles of Cα-C—N, C—N—Cα and N—Cα-C of each residue, the Cα-Cα distance, and the Cβ-Cβ distance, as shown in FIG. 4.

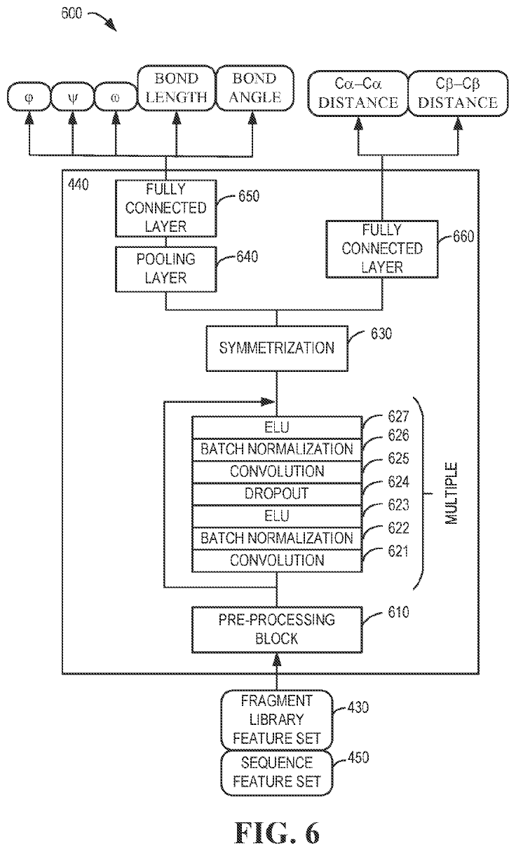

Reference is made to FIG. 6, which shows a schematic diagram of a process 600 of predicting structural properties of the protein by using the property predictor 440 according to some implementations of the present disclosure. The fragment library feature set 430 and the sequence feature set 450 inputted to the property predictor 440 may first be processed by a pre-processing block 610. As an example, the pre-processing block 610 may comprise a two-dimensional convolutional layer, a batch normalization layer and an ELU activation layer, etc. A two-dimensional residual neural network with a plurality of (e.g., residual blocks follows the pre-processing block 610. As an example, FIG. 6 shows that each residual block may comprise two convolutional layers 621, 625 and two ELU activation layers 623, 627. In addition, to prevent overfitting, batch normalization layers 622, 626 may be adopted after the convolutional layers 621, 625 and a dropout layer 624, with a dropout rate of e.g., 0.15, may be used.

A symmetrization operation 630 is performed on the output of the residual network. The output of the symmetrization operation 630 is then inputted into two respective branches to predict different structural properties. The left branch shown in FIG. 6 comprises a pooling layer 640, which converts the two-dimensional feature map outputted by the symmetrization operation 630 into a one-dimensional feature vector. Subsequently, the one-dimensional feature vector is inputted into a fully connected layer 650, which outputs 1D structural properties of each residue of the target protein, e.g., torsion angles φ, ψ, ω, bond lengths and bond angles between continuous backbone atoms. The right branch shown in FIG. 6 directly predicts the Cα-Cα distance and the Cβ-Cβ distance using the fully connected layer 660. In this example, the property predictor 440 may be implemented as a multi-task predictor to simultaneously predict a plurality of structural properties of the target protein.

Reference is made back to FIG. 4. In training, the feature encoder 420 and the property predictor 440 may be jointly trained with a training dataset. The sum of the mean absolute errors (MAE) of all the output structural properties may be used as the loss function.

Description has been presented above for example implementations of predicting structural properties of a protein by using structural information of a fragment library. In such implementations, structural features of fragments in the fragment library are implicitly represented using features generated by the feature encoder. Such implicit features derived from the fragment library are subsequently inputted into the property predictor to predict one or more structural properties of the protein. Compared with the method which does not utilize implicit features derived from the fragment library, the method utilizing implicit features derived from the fragment library may improve the accuracy of structural property prediction

### Example Method and Example Implementations

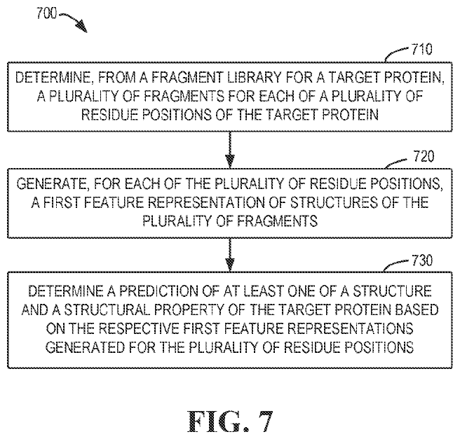

FIG. 7 shows a flowchart of a method 500 for protein structure prediction according to some implementations of the present disclosure. The method 700 may be implemented by the computing device 100, e.g., may be implemented at the prediction module 122 in the memory 120 of the computing device 100.

As shown in FIG. 7, at block 710, the computing device 100 determines, from a fragment library for a target protein, a plurality of fragments for each of a plurality of residue positions of the target protein. Each fragment comprises a plurality of amino acid residues.

In some implementations, to determine the plurality of fragments, the computing device 100 may determine an initial fragment assigned by the fragment library to each residue position; and generate from the initial fragment fragments with a predetermined number of residues as the plurality of fragments.

At block 720, the computing device 100 generates for the each residue position a first feature representation of structures of the plurality of fragments. For example, the computing device 100 may determine Gaussian distributions of structural properties at the each residue position, or generate the fragment library feature set 430. At block 730, the computing device 100 determines a prediction of at least one of a structure and a structural property of the target protein based on the respective first feature representations generated for the plurality of residue positions.

In some implementations, to generate the first feature representation, the computing device 100 may determine, for each residue position, a property value of a structural property of each fragment based on structures of the plurality of fragments; and determine, based on property values of the structural property of the plurality of fragments, a probability distribution of the structural property at each residue position as the first feature representation. In some implementations, to determine the prediction of the structure of the target protein, the computing device 100 may generate a potential function corresponding to the structural property based on the respective probability distributions at the plurality of residue positions; determine, based on the potential function, a target function of a structure prediction model for predicting a structure of a protein; and determine the prediction of the structure of the target protein by minimizing the target function according to the structure prediction model.

In some implementations, the structural property may comprise at least one of: an angle between atoms of different types, angles between atoms of the same type, or distances between atoms of the same type.

In some implementations, to generate the first feature representation, the computing device 100 may determine, for each residue position, a plurality of structural properties of each fragment based on structures of the plurality of fragments; and determine the first feature representation by encoding the plurality of structural properties of each fragment of the plurality of fragments according to a trained feature encoder. In some implementations, to determine the prediction of the structural property of the target protein, the computing device 100 may determine a second feature representation of an amino acid sequence of the target protein, the amino acid sequence indicating a residue type at each of the plurality of residue positions; and determine the prediction of the structural property of the target protein based on the respective first feature representations and second feature representations determined for the plurality of residue positions according to a trained property predictor.

In some implementations, the method 700 further comprises: for each of a plurality of reference fragment libraries built for a reference protein based on different algorithms, determining reference property values of a structural property of a plurality of reference fragments assigned by each reference fragment library to a reference residue position of the reference protein; determining a true property value of the structural property of the reference protein at the reference residue position; determining a difference between the reference property values and the true property value; and selecting, based on the respective differences determined for the plurality of reference fragment libraries, a target algorithm from the different algorithms for building the fragment library for the target protein.

As seen from the above description, the solution for protein structure prediction according to the implementations of the present disclosure can utilize structural information of the fragment library to complement and complete information used in protein structure prediction. In this way, the accuracy of protein structure prediction may be improved.

Some example implementations of the present disclosure are listed below.

In one aspect, the present disclosure provides a computer-implemented method. The method comprises: determining, from a fragment library for a target protein, a plurality of fragments for each of a plurality of residue positions of the target protein, each fragment comprising a plurality of amino acid residues; generating, for each of the plurality of residue positions, a first feature representation of structures of the plurality of fragments; and determining a prediction of at least one of a structure and a structural property of the target protein based on the respective first feature representations generated for the plurality of residue positions.

In some implementations, generating the first feature representation comprises: determining, for each residue position, a property value of a structural property of each fragment based on structures of the plurality of fragments; and determining, based on property values of the structural property of the plurality of fragments, a probability distribution of the structural property at the each residue position as the first feature representation.

In some implementations, determining the prediction of the structure of the target protein comprises: generating a potential function corresponding to the structural property based on the respective probability distributions at the plurality of residue positions; determining, based on the potential function, a target function of a structure prediction model for predicting a structure of a protein; and determining the prediction of the structure of the target protein by minimizing the target function according to the structure prediction model.

In some implementations, determining the plurality of fragments comprises: determining initial fragments assigned by the fragment library to each residue position; and generating, from the initial fragments, fragments with a predetermined number of residues as the plurality of fragments.

In some implementations, the structural property comprises at least one of: an angle between atoms of different types, an angle between atoms of the same type, or a distance between atoms of the same type.

In some implementations, generating the first feature representation comprises: determining, for each residue position, a plurality of structural properties of each fragment based on structures of the plurality of fragments; and determining the first feature representation by encoding the plurality of structural properties of each fragment of the plurality of fragments according to a trained feature encoder.

In some implementations, determining the prediction of the structural property of the target protein comprises: determining a second feature representation of an amino acid sequence of the target protein, the amino acid sequence indicating a reside type at each of the plurality of residue positions; and determining the prediction of the structural property of the target protein based on the respective first feature representations and second feature representations determined for the plurality of residue positions according to a trained property predictor.

In some implementations, the structural property comprises at least one of: an angle between atoms of different types, an angle between atoms of the same type, or a distance between atoms of the same type.

In some implementations, the method further comprises: for each of a plurality of reference fragment libraries built for a reference protein based on different algorithms, determining reference property values of a structural property of a plurality of reference fragments assigned by each reference fragment library to a reference residue position of the reference protein; determining a true property value of the structural property of the reference protein at the reference residue position; determining a difference between the reference property values and the true property value; and selecting, based on the respective differences determined for the plurality of reference fragment libraries, a target algorithm from the different algorithms for building the fragment library for the target protein.

In another aspect, the present disclosure provides an electronic device. The electronic device comprises: a processing unit; and a memory coupled to the processing unit and comprising instructions stored thereon which, when executed by the processing unit, cause the device to perform acts comprising: determining, from a fragment library for a target protein, a plurality of fragments for each of a plurality of residue positions of the target protein, each fragment comprising a plurality of amino acid residues; generating, for each of the plurality of residue positions, a first feature representation of structures of the plurality of fragments; and determining a prediction of at least one of a structure and a structural property of the target protein based on the respective first feature representations generated for the plurality of residue positions.

In some implementations, generating the first feature representation comprises: determining, for each residue position, a property value of a structural property of each fragment based on structures of the plurality of fragments; and determining, based on property values of the structural property of the plurality of fragments, a probability distribution of the structural property at the each residue position as the first feature representation.

In some implementations, determining the prediction of the structure of the target protein comprises: generating a potential function corresponding to the structural property based on the respective probability distributions at the plurality of residue positions; determining, based on the potential function, a target function of a structure prediction model for predicting a structure of a protein; and determining the prediction of the structure of the target protein by minimizing the target function according to the structure prediction model.

In some implementations, determining the plurality of fragments comprises: determining initial fragments assigned by the fragment library to each residue position; and generating, from the initial fragments, fragments with a predetermined number of residues as the plurality of fragments.

In some implementations, the structural property comprises at least one of: an angle between atoms of different types, an angle between atoms of the same type, or a distance between atoms of the same type.

In some implementations, generating the first feature representation comprises: determining, for each residue position, a plurality of structural properties of each fragment based on structures of the plurality of fragments; and determining the first feature representation by encoding the plurality of structural properties of each fragment of the plurality of fragments according to a trained feature encoder.

In some implementations, determining the prediction of the structural property of the target protein comprises: determining a second feature representation of an amino acid sequence of the target protein, the amino acid sequence indicating a reside type at each of the plurality of residue positions; and determining the prediction of the structural property of the target protein based on the respective first feature representations and second feature representations determined for the plurality of residue positions according to a trained property predictor.

In some implementations, the structural property comprises at least one of: an angle between atoms of different types, an angle between atoms of the same type, or a distance between atoms of the same type.

In some implementations, the acts further comprise: for each of a plurality of reference fragment libraries built for a reference protein based on different algorithms, determining reference property values of a structural property of a plurality of reference fragments assigned by each reference fragment library to a reference residue position of the reference protein; determining a true property value of the structural property of the reference protein at the reference residue position; determining a difference between the reference property values and the true property value; and selecting, based on the respective differences determined for the plurality of reference fragment libraries, a target algorithm from the different algorithms for building the fragment library for the target protein.

In a further aspect, the present disclosure provides a computer program product being tangibly stored in a non-transitory computer storage medium and comprising machine-executable instructions which, when executed by a device, causing the device to perform the method of the above aspect.

In yet a further aspect, the present disclosure provides a computer-readable medium having machine-executable instructions stored thereon which, when executed by a device, causes the device to perform the method of the above aspect.

The functionality described herein can be performed, at least in part, by one or more hardware logic components. For example, and without limitation, illustrative types of hardware logic components that can be used include Field-Programmable Gate Arrays (FPGAs), Application-specific Integrated Circuits (ASICs), Application-specific Standard Products (ASSPs), System-on-a-chip systems (SOCs), Complex Programmable Logic Devices (CPLDs), and the like.

Program code for carrying out methods of the present disclosure may be written in any combination of one or more programming languages. These program codes may be provided to a processor or controller of a general purpose computer, a special purpose computer, or other programmable data processing apparatus, such that the program codes, when executed by the processor or controller, cause the functions/operations specified in the flowcharts and/or block diagrams to be implemented. The program code may execute entirely on a machine, partly on the machine, as a stand-alone software package, partly on the machine and partly on a remote machine or entirely on the remote machine or a server.

In the context of this present disclosure, a machine-readable medium may be any tangible medium that may contain, or store a program for use by or in connection with an instruction execution system, apparatus, or device. The machine-readable medium may be a machine-readable signal medium or a machine-readable storage medium. A machine-readable medium may include, but is not limited to, an electronic, magnetic, optical, electromagnetic, infrared, or semiconductor system, apparatus, or device, or any suitable combination of the foregoing. More specific examples of the machine-readable storage medium would include an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing.

Further, although operations are depicted in a particular order, it should be understood that the operations are required to be executed in the particular order shown or in a sequential order, or all operations shown are required to be executed to achieve the expected results. In certain circumstances, multitasking and parallel processing may be advantageous. Likewise, while several specific implementation details are contained in the above discussions, these should not be construed as limitations on the scope of the present disclosure. Certain features that are described in the context of separate implementations may also be implemented in combination in a single implementation. Conversely, various features that are described in the context of a single implementation may also be implemented in multiple implementations separately or in any suitable sub-combination.

Although the subject matter has been described in language specific to structural features and/or methodological acts, it is to be understood that the subject matter specified in the appended claims is not necessarily limited to the specific features or acts described above. Rather, the specific features and acts described above are disclosed as example forms of implementing the claims.

1. A computer-implemented method, comprising:
determining, from a fragment library for a target protein, a plurality of fragments for each of a plurality of residue positions of the target protein, each fragment comprising a plurality of amino acid residues;
generating, for each of the plurality of residue positions, a first feature representation of structures of the plurality of fragments; and
determining a prediction of at least one of a structure and a structural property of the target protein based on the respective first feature representations generated for the plurality of residue positions.

2. The method of claim 1, wherein generating the first feature representation comprises:
determining, for each residue position, a property value of a structural property of each fragment based on structures of the plurality of fragments; and
determining, based on property values of the structural property of the plurality of fragments, a probability distribution of the structural property at the each residue position as the first feature representation.

3. The method of claim 2, wherein determining the prediction of the structure of the target protein comprises:
generating a potential function corresponding to the structural property based on the respective probability distributions at the plurality of residue positions;
determining, based on the potential function, a target function of a structure prediction model for predicting a structure of a protein; and
determining the prediction of the structure of the target protein by minimizing the target function according to the structure prediction model.

4. The method of claim 2, wherein determining the plurality of fragments comprises:
determining initial fragments assigned by the fragment library to each residue position; and
generating, from the initial fragments, fragments with a predetermined number of residues as the plurality of fragments.

5. The method of claim 2, wherein the structural property comprises at least one of:
an angle between atoms of different types,
an angle between atoms of the same type, or
a distance between atoms of the same type.

6. The method of claim 1, wherein generating the first feature representation comprises:
determining, for each residue position, a plurality of structural properties of each fragment based on structures of the plurality of fragments; and
determining the first feature representation by encoding the plurality of structural properties of each fragment of the plurality of fragments according to a trained feature encoder.

7. The method of claim 6, wherein determining the prediction of the structural property of the target protein comprises:
determining a second feature representation of an amino acid sequence of the target protein, the amino acid sequence indicating a reside type at each of the plurality of residue positions; and
determining the prediction of the structural property of the target protein based on the respective first feature representations and second feature representations determined for the plurality of residue positions according to a trained property predictor.

8. The method of claim 1, further comprising:
for each of a plurality of reference fragment libraries built for a reference protein based on different algorithms,
determining reference property values of a structural property of a plurality of reference fragments assigned by each reference fragment library to a reference residue position of the reference protein;
determining a true property value of the structural property of the reference protein at the reference residue position;
determining a difference between the reference property values and the true property value; and
selecting, based on the respective differences determined for the plurality of reference fragment libraries, a target algorithm from the different algorithms for building the fragment library for the target protein.

9. An electronic device, comprising:
a processing unit; and
a memory coupled to the processing unit and comprising instructions stored thereon which, when executed by the processing unit, cause the electronic device to perform acts comprising:
determining, from a fragment library for a target protein, a plurality of fragments for each of a plurality of residue positions of the target protein, each fragment comprising a plurality of amino acid residues;
generating, for each of the plurality of residue positions, a first feature representation of structures of the plurality of fragments; and
determining a prediction of at least one of a structure and a structural property of the target protein based on the respective first feature representations generated for the plurality of residue positions.

10. The device of claim 9, wherein generating the first feature representation comprises:
determining, for each residue position, a property value of a structural property of each fragment based on structures of the plurality of fragments; and
determining, based on property values of the structural property of the plurality of fragments, a probability distribution of the structural property at the each residue position as the first feature representation.

11. The device of claim 10, wherein determining the prediction of the structure of the target protein comprises:
generating a potential function corresponding to the structural property based on the respective probability distributions at the plurality of residue positions;
determining, based on the potential function, a target function of a structure prediction model for predicting a structure of a protein; and
determining the prediction of the structure of the target protein by minimizing the target function according to the structure prediction model.

12. The device of claim 10, wherein determining the plurality of fragments comprises:
determining initial fragments assigned by the fragment library to each residue position; and
generating, from the initial fragments, fragments with a predetermined number of residues as the plurality of fragments.

13. The device of claim 9, wherein generating the first feature representation comprises:
determining, for each residue position, a plurality of structural properties of each fragment based on structures of the plurality of fragments; and
determining the first feature representation by encoding the plurality of structural properties of each fragment of the plurality of fragments according to a trained feature encoder.

14. The device of claim 13, wherein determining the prediction of the structural property of the target protein comprises:
determining a second feature representation of an amino acid sequence of the target protein, the amino acid sequence indicating a reside type at each of the plurality of residue positions; and
determining the prediction of the structural property of the target protein based on the respective first feature representations and second feature representations determined for the plurality of residue positions according to a trained property predictor.

15. A computer program product being tangibly stored in a computer storage medium and comprising machine-executable instructions which, when executed by a device, cause the device to perform acts comprising:
determining, from a fragment library for a target protein, a plurality of fragments for each of a plurality of residue positions of the target protein, each fragment comprising a plurality of amino acid residues;
generating, for each of the plurality of residue positions, a first feature representation of structures of the plurality of fragments; and
determining a prediction of at least one of a structure and a structural property of the target protein based on the respective first feature representations generated for the plurality of residue positions.
